(19) United States
(12) Patent Application Publication
(10) Pub. No.: US 2015/0119677 A1
(43) Pub. Date: Apr. 30, 2015

(54) COMBINATION OF ARTICLE OF CLOTHING AND ECG ELECTRODES

(71) Applicant: Weiming LIU, Wuxi (CN)

(72) Inventor: Weiming LIU, Wuxi (CN)

(21) Appl. No.: 14/220,108

(22) Filed: Mar. 19, 2014

(30) Foreign Application Priority Data
Oct. 24, 2013 (CN) 201310507592.1
Oct. 24, 2013 (CN) 201310511358.6

(51) Int. Cl. A61B 5/0408; A61B 5/00

(52) U.S. Cl. CPC A61B 5/04085; A61B 5/6805
USPC 600/389

(57) ABSTRACT

A combination of an article of clothing and ECG electrodes, the combination including a front piece and a back piece. The front piece includes an inner lining and an outer lining. Ten front-end modules of active electrodes, ten lead wires, and an electrical plug are disposed on the outer surface of the inner lining for the purpose of collecting electrical signals. The ten front-end modules are connected to the electrical plug via the ten lead wires, respectively. The front-end modules are riveted on the inner lining by bolts. The outer lining includes a pocket including a through hole. The electrical plug passes through the through hole and is inserted into a Holter system placed in the pocket. A plurality of female buckles for buckling ECG electrodes are disposed on an inner surface of the inner lining.

### CROSS-REFERENCE TO RELATED APPLICATIONS

Pursuant to 35 U.S.C. §119 and the Paris Convention Treaty, this application claims the benefit of Chinese Patent Application No. 201310507592.1 filed Oct. 24, 2013 and of Chinese Patent Application No. 201310511358.6 filed Oct. 24, 2013. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 14781 Memorial Drive, Suite 1319, Houston, Tex. 77079.

### BACKGROUND OF THE INVENTION

1. Field of the Invention

This invention relates to the field of medical diagnosis, and more particularly to a combination of an article of clothing and ECG electrodes.

2. Description of the Related Art

Dynamic electrocardiogram apparatus such as a Holter system can be used to continuously record electrocardiogram over 24 hours for patients. By so doing, the detection rates of patients with sporadic and non-persistent arrhythmia can be improved. Holter employs one-time paste electrodes which are apt to drop off or have poor contact with skin in daily clinical use due to such factors as body activities, sweat, and grease and so on. Meanwhile, with the lead wires exposed, electrodes and the lead wires easily drop off due to daily body activities. Therefore, the wearing way of conventional dynamic electrocardiogram apparatus severely limits the daily life and activities of the user, causing much inconvenience and discomfort.

### SUMMARY OF THE INVENTION

In view of the above-described problems, it is one objective of the invention to provide a combination of an article of clothing and ECG electrodes. The combination can achieve the long-term dynamic monitoring of ECG data of the patients in their normal daily work or life, especially in motion state such as walking and running.

To achieve the above objective, in accordance with one embodiment of the invention, there is provided a combination of an article of clothing and ECG electrodes, comprising a front piece, the front piece comprising an inner lining and an outer lining; and a back piece. The ten front-end modules of active electrodes, ten lead wires, and an electrical plug for collecting electrical signals are disposed on an outer surface of the inner lining. The ten front-end modules of active electrodes are connected to the electrical plug via the ten lead wires, respectively. The ten front-end modules of active electrodes are riveted on the inner lining by bolts. The outer lining comprises a pocket comprising a through hole. The electrical plug passes through the through hole and is inserted into a Holter system placed in the pocket. A plurality of female buckles for buckling ECG electrodes are disposed on an inner surface of the inner lining. Both sides of the article of clothing where the front piece and the back piece are sewn are provided with a first zipper for adjusting tightness.

In a class of this embodiment, the article of clothing can be worn on the upper body of a wearer.

In a class of this embodiment, the article of clothing is a cardigan, a jacket, a crew neck, a sweater, a jersey, a jumper, a polo neck, a turtleneck, a pullover, a sweatshirt, a turtleneck, a vest, a coat, a robe, etc.

In a class of this embodiment, the article of clothing comprises a neck area and an abdomen area, and a second zipper extending from the neck area to the abdomen area is disposed in a middle line of the front piece of the article of clothing.

In a class of this embodiment, both the inner lining of the front piece and the back piece are made of two different kinds of cloth.

In a class of this embodiment, the two different kinds of cloth comprise non-elastic cloth and elastic cloth.

In a class of this embodiment, an anti-skid plastic elastic band is disposed at a lower part of an inner side of both the front piece and the back piece of the article of clothing.

In a class of this embodiment, each of the ten front-end modules of active electrodes comprises a first elastic pad, a signal processing PCB, and a metal mask; the bolts pass through the signal processing PCB, the elastic pad, and the inner lining of the front piece, and then are connected to the female buckles; the metal mask is filled with silica gel which covers the signal processing PCB; and a second elastic pad is disposed at an outer side of the metal mask whereby increasing the tight pressure of electrodes on the human body.

In a class of this embodiment, the ECG electrode comprises an electrode cap, an anti-skid washer, a conductive pad, and an electrode buckle; the electrode cap is made from conductive gel or conductive cloth mixed with elastic materials; the anti-skid washer is made from soft gel; the conductive pad is made from conductive materials; the electrode buckle is formed by stamping conductive metals and fixed at the center of the conductive pad.

In a class of this embodiment, an EGC machine is connected to the ECG electrodes.

In a class of this embodiment, an EGC machine is connected to the piece of clothing.

### BRIEF DESCRIPTION OF THE DRAWINGS

FIG. 1 is a schematic diagram of an inner lining of a front piece of a combination of an article of clothing and ECG electrodes in accordance with one embodiment of the invention;

FIG. 2 is a position distribution diagram of ECG electrodes in a Holter system in accordance with one embodiment of the invention;

FIG. 3 shows a connection of front-end modules of active electrodes, lead wires, and an electrical plug in accordance with one embodiment of the invention;

FIG. 4 is a schematic diagram of an outer lining of a front piece outer of a combination of an article of clothing and ECG electrodes in accordance with one embodiment of the invention;

FIG. 5 is a schematic diagram of a front piece of a combination of an article of clothing and ECG electrodes in accordance with one embodiment of the invention;

FIG. 6 is a schematic diagram of a back piece of a combination of an article of clothing and ECG electrodes in accordance with one embodiment of the invention;

FIG. 7 is a front view of a combination of an article of clothing and ECG electrodes in accordance with one embodiment of the invention;

FIG. 8 is a front perspective view of a combination of an article of clothing and ECG electrodes in accordance with one embodiment of the invention;

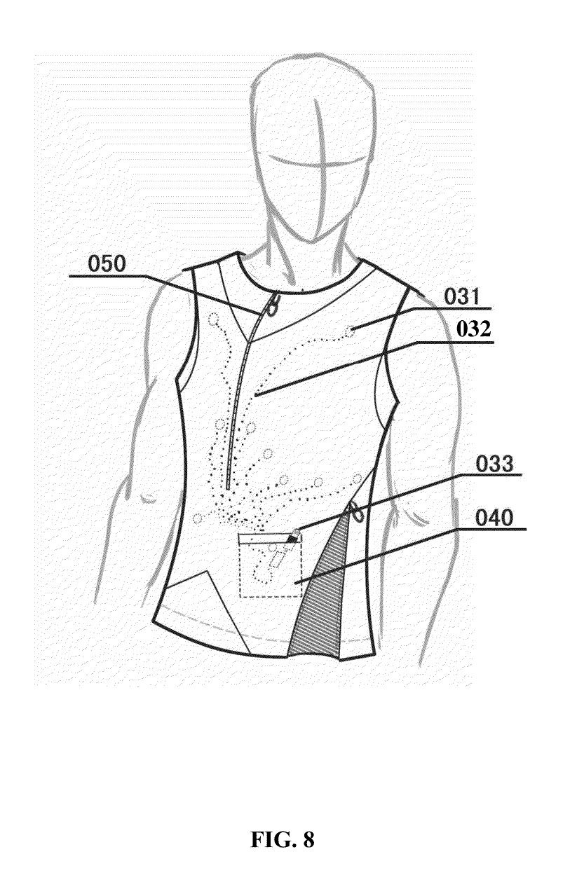

FIG. 9 is an exploded view of a front-end module of an active electrode of a combination of an article of clothing and ECG electrodes in accordance with one embodiment of the invention;

FIG. 10 is an exploded view of an ECG electrode of a combination of an article of clothing and ECG electrodes in accordance with one embodiment of the invention; and

FIG. 11 shows an assembly of an ECG electrode and a female buckle of a combination of an article of clothing and ECG electrodes in accordance with one embodiment of the invention.

### DETAILED DESCRIPTION OF THE PREFERRED EMBODIMENTS

The invention provides a combination of an article of clothing and ECG electrodes, comprising a front piece and a back piece. The front piece comprises an inner lining and an outer lining. Ten front-end modules of active electrodes 031, ten lead wires 032, and an electrical plug 033 for collecting electrical signals are disposed on an outer surface of the inner lining. The ten front-end modules of active electrodes are connected to the electrical plug via the ten lead wires, respectively. The ten front-end modules of active electrodes are riveted on the inner lining by bolts. The outer lining comprises a pocket 040 comprising a through hole. The electrical plug passes through the through hole and is inserted into a Holter system placed in the pocket 040. A plurality of female buckles 090 for buckling ECG electrodes are disposed on an inner surface of the inner lining. Both sides of the combination of an article of clothing and ECG electrodes where the front piece and the back piece are sewn are provided with a first zipper for adjusting tightness.

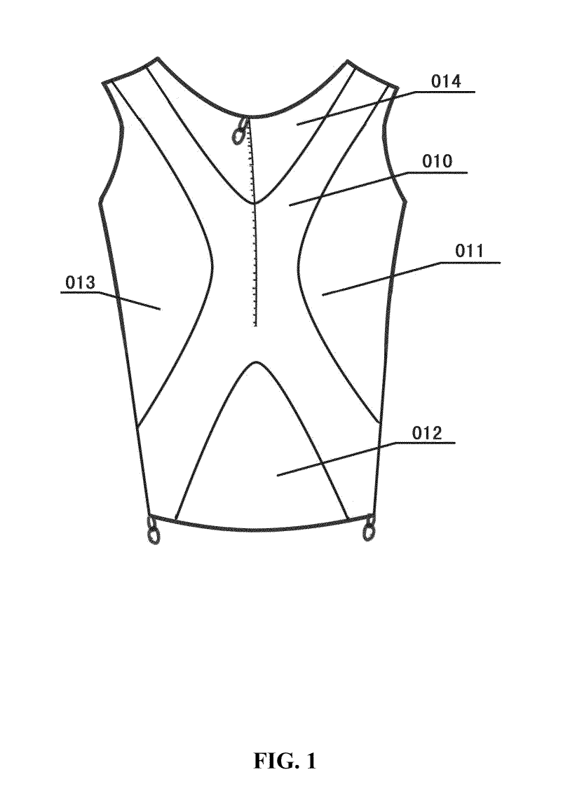

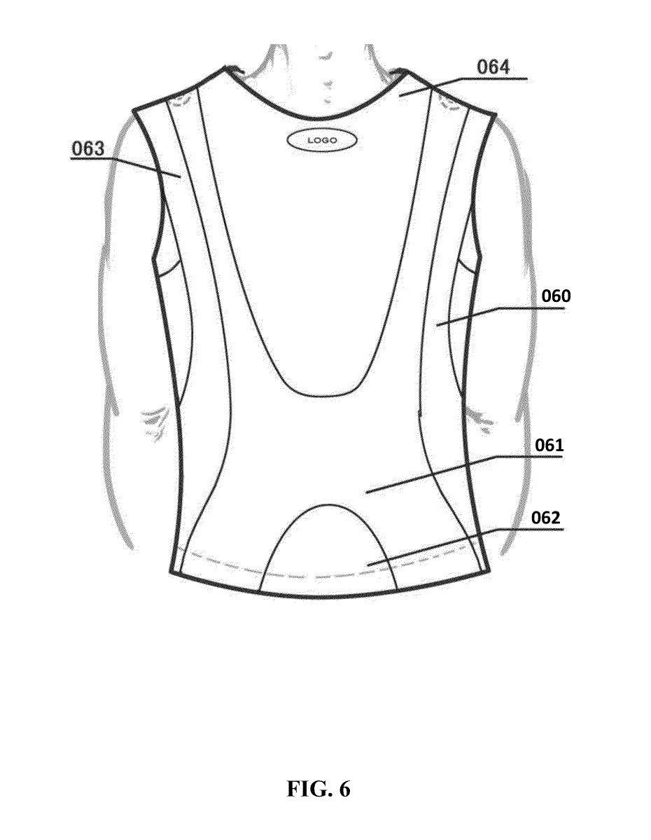

As shown in FIG. 1, the inner lining of the front piece of the combination of an article of clothing and ECG electrodes is made of two different kinds of cloth. The X-shaped part 010 is non-elastic cloth, and parts 011, 012, 013 and 014 are all highly elastic. As shown in FIG. 6, the back piece of the combination is also made of two different kinds of cloth. The X-shaped part 060 is non-elastic cloth, and parts 061, 062, 063 and 064 are all highly elastic. The design is a combination of ergonomics, sports medicine and the advanced textile techniques, which effectively helps to offer the required tightness at various body parts while ECG detection and measurement is being done, and ensures the ECG electrodes 100 in close contact with the skin and eliminates the potential influence of the moving of electrodes and the muscle on ECG measurements.

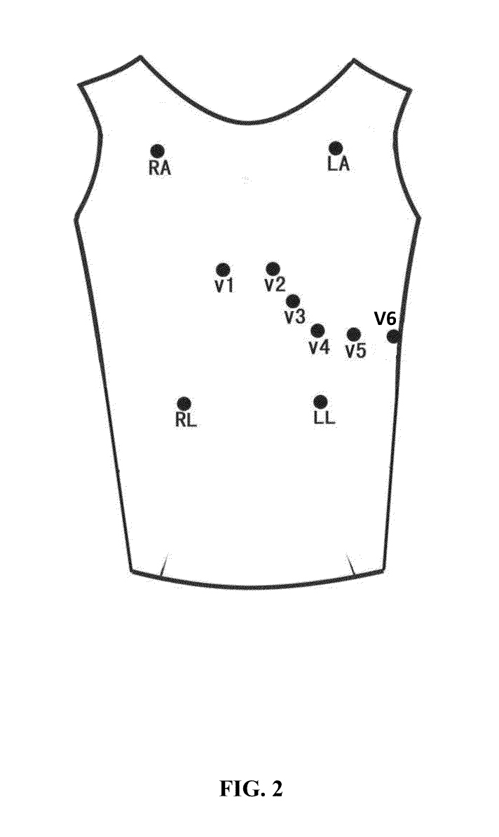

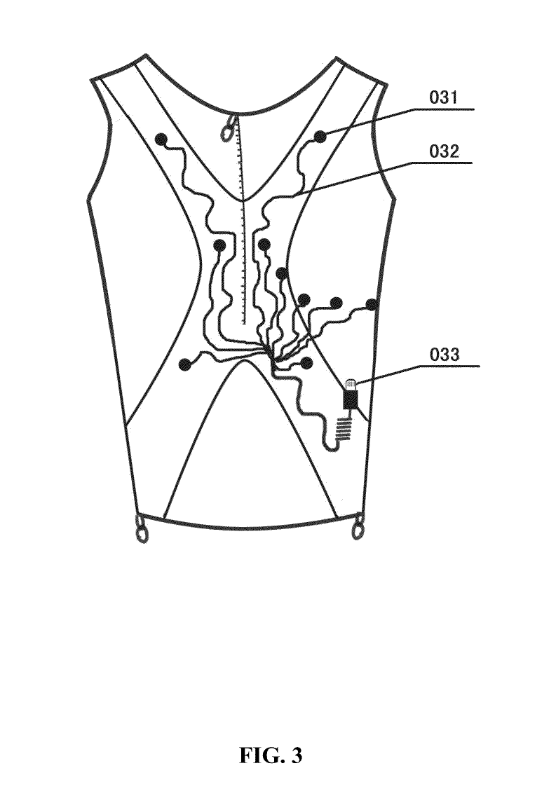

As shown in FIGS. 2 and 3, according to the positions of the ECG electrodes RA, LA, RL, LL, V1, V2, V3, V4, V5 and V6 in an ECG apparatus with 12 lead wires, ten front-end modules of active electrodes 031, ten lead wires 032, and an electrical plug 033 for collecting electrical signals are disposed on an outer surface of the inner lining. The front-end modules of active electrodes 031 are fixed at the inner lining of the front piece of the pullover via bolts 093. A plurality of female buckles 090 for buckling ECG electrodes are disposed on an inner surface (that is, in contact with the body) of the inner lining. The ten front-end modules of active electrodes 031, the ten lead wires 032, and the electrical plug 033 for collecting electrode signals are disposed on an outer surface (i.e., not in contact with the body) of the inner lining.

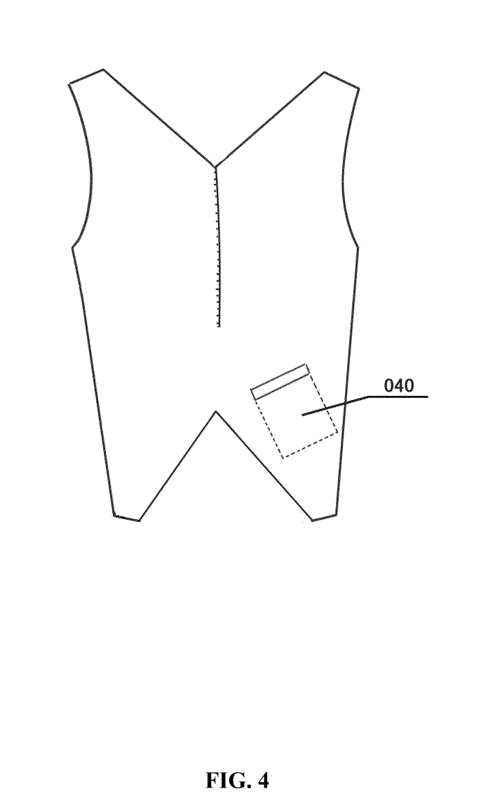

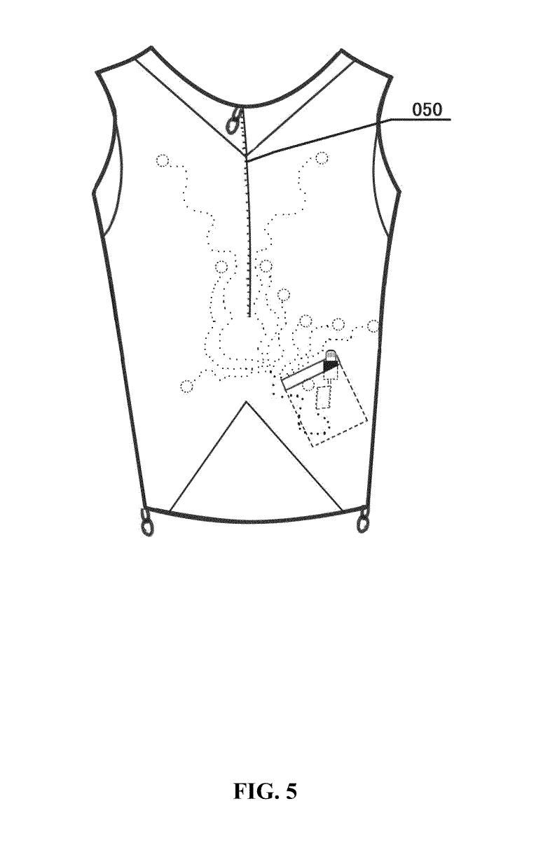

As shown in FIG. 4, the outer lining of the front piece, made of highly elastic cloth, is used to hide and protect the components disposed in the inner lining, namely, the front-end modules of active electrodes 031, the lead wires 032 and the electrical plug 033. A dynamic electrocardiogram apparatus (a Holter system) can also be placed in the outer lining of the front piece. As shown in FIGS. 4 and 5, a pocket 040 comprising a through hole is disposed on the outer lining of the front piece of the pullover for receiving the Holter system. The electrical plug 033 passes through the through hole and is inserted into the Holter system.

As shown in FIG. 5, the inner lining of the front piece comprising the front-end modules of active electrodes 031, the lead wires 032 and the electrical plug 033 is sewn with the outer lining comprising the pocket 040 to form the front piece of the piece of clothing. The female buckle for buckling ECG electrodes is disposed on the inner surface of the inner lining of the front piece to connect to ECG electrodes 100. The front-end modules of active electrodes 031 and the lead wires 032 are arranged between the inner lining and the outer lining of the front piece. The electrical plug 033 for collecting electrical signals is put in the pocket 040 of the front piece of the piece of clothing. The combination of an article of clothing and ECG electrodes comprises a neck area and an abdomen area, and a second zipper 050 extending from the neck area to the abdomen area is disposed in a middle line of the front piece of the combination, which facilitates the installation of the ECG electrodes.

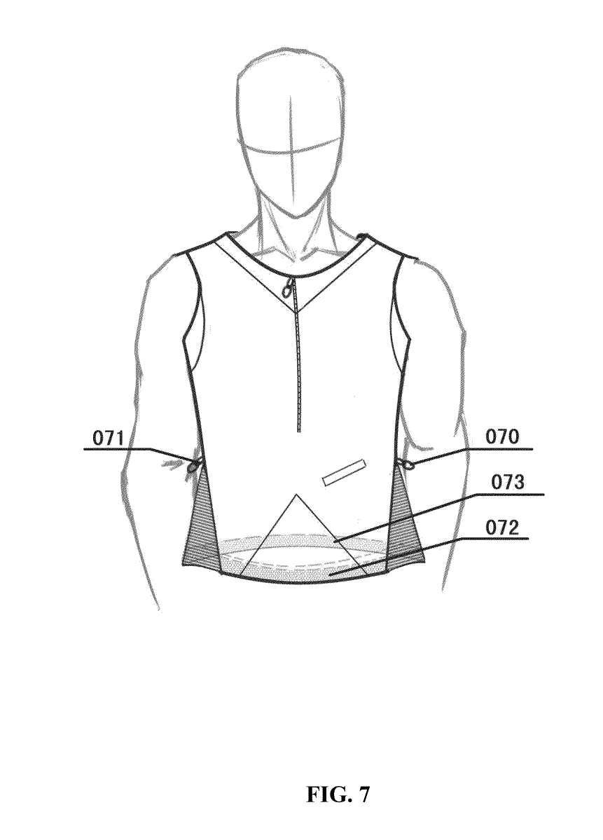

As shown in FIG. 7, both sides of the combination of an article of clothing and ECG electrodes where the front piece and the back piece are sewn are provided with first zippers 070, 071 for adjusting tightness. When the first zippers are fastened, the combination is closely attached to the abdomen.

As shown in FIG. 7, anti-skid plastic elastic bands 072, 073 are disposed at a lower part of an inner side of both the front piece and the back piece of the combination of an article of clothing and ECG electrodes. Thus, after both the side zippers are fastened, the required tightness is produced whereby preventing the upward sliding of the piece of clothing.

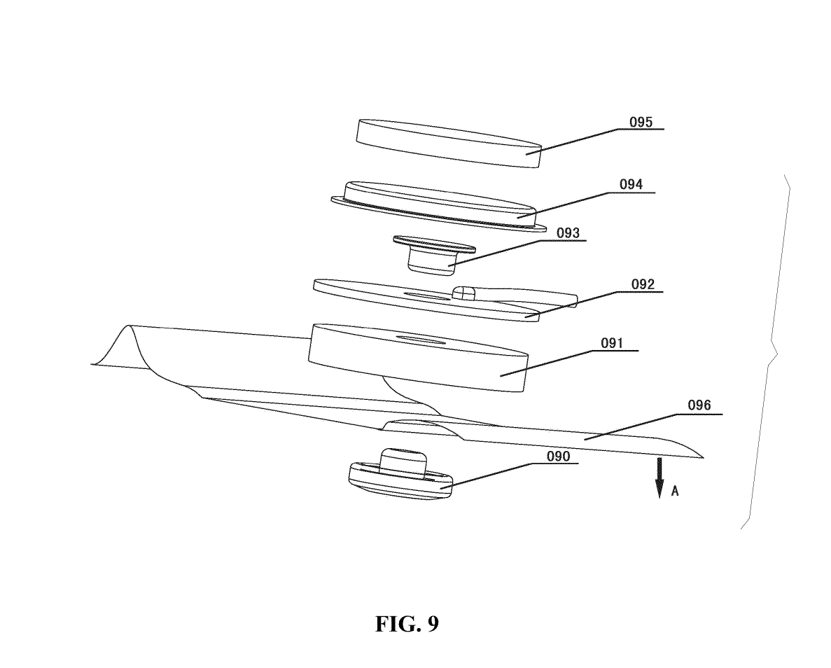

As shown in FIG. 9, the front-end modules of active electrodes 031 comprise an elastic pad 091, a signal processing PCB 092, and a metal mask 094. The bolts 093 pass through the signal processing PCB 092, the elastic pad 091, and the inner lining 096 of the front piece, and then are connected to the female buckles 090. The direction ‘A’ in FIG. 9 indicates the direction of the skin. The metal mask 094 is filled with silica gel which covers the signal processing PCB 092, whereby ensuring the anti-electromagnetic interference and waterproof properties of the signal processing PCB. A second elastic pad 095 is disposed at an outer side of the metal mask 094 whereby increasing the tight pressure of electrodes on the human body.

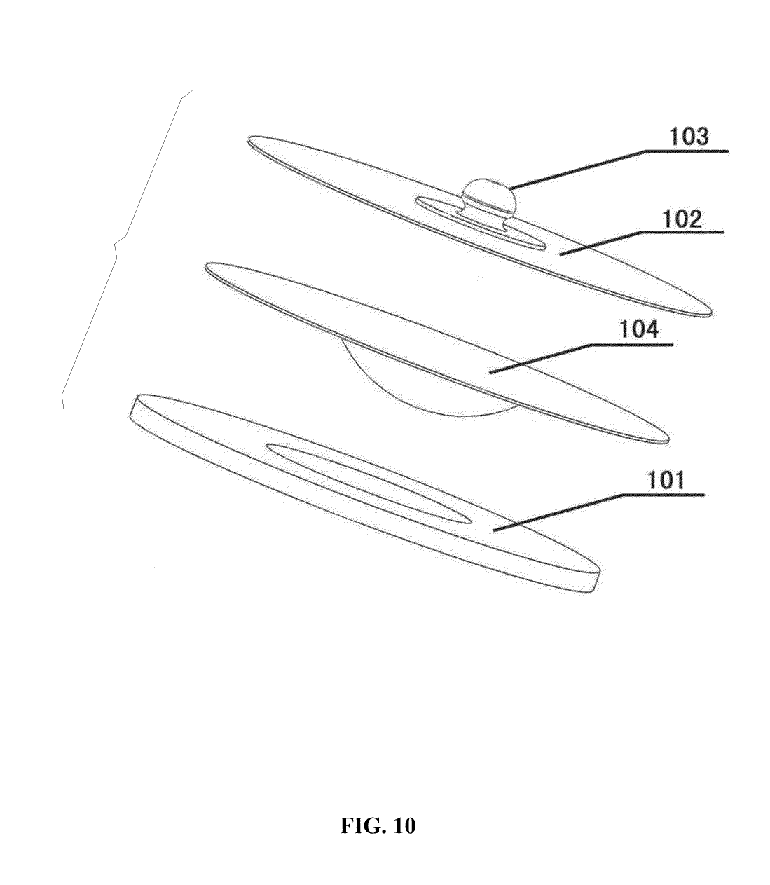

As shown in FIG. 10, the ECG electrode 100 comprises an electrode cap 104, an anti-skid washer 101, a conductive pad 102 and an electrode buckle 103. The electrode cap 104 is made from conductive gel or conductive cloth mixed with elastic materials such as sponge. The hump of the electrode cap 104 is in contact with the body. The anti-skid washer 101 is made from some soft gel and does not slide easily when in contact with the skin thereby preventing the ECG electrode from sliding. The conductive pad 102 is made from conductive materials and the electrode buckle 103 is formed by stamping conductive metals and fixed at the center of the conductive pad. The electrode cap 104, the anti-skid washer 101, and the conductive pad 102 preloaded with the electrode buckle 103 are stuck and pressed to form the ECG electrode 100.

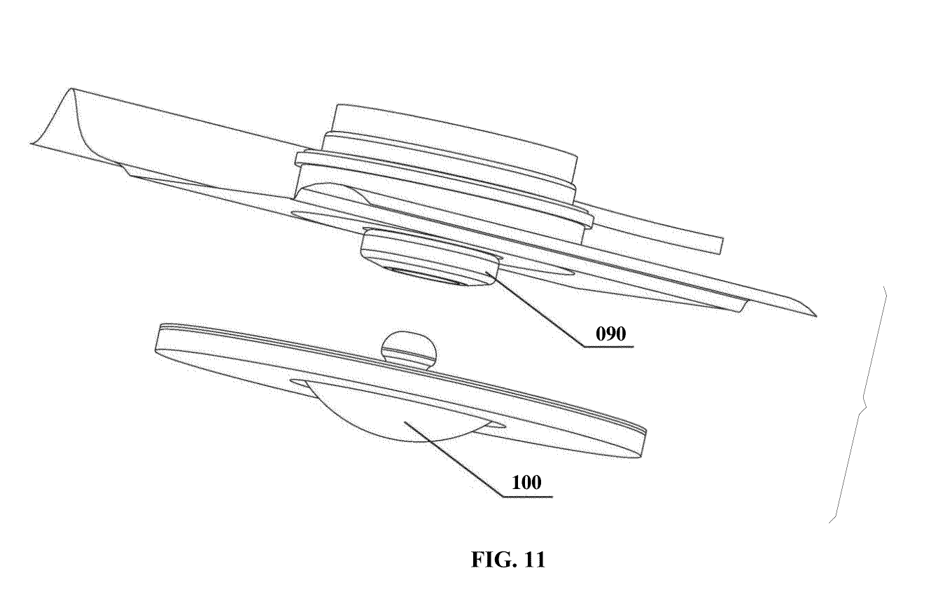

As shown in FIG. 11, the ECG electrode cooperates with the female buckle. It's very easy to install, remove, and change the electrodes as needed. In use, the ECG electrode 100 is first assembled with the female buckle 110 and then put on the piece of clothing. Optionally, put on the piece of clothing first, unfasten the second zipper 050, connect the electrode 100 to the female buckle 110, and finally fasten the second zipper 050.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A combination of an article of clothing and ECG electrodes, the combination comprising:
a) a front piece, the front piece comprising an inner lining and an outer lining; and
b) a back piece; wherein
ten front-end modules of active electrodes, ten lead wires, and an electrical plug for collecting electrical signals are disposed on an outer surface of the inner lining;
the ten front-end modules of active electrodes are connected to the electrical plug via the ten lead wires, respectively;
the ten front-end modules of active electrodes are riveted on the inner lining by bolts;
the outer lining comprises a pocket comprising a through hole;
the electrical plug passes through the through hole and is inserted into a Holter system placed in the pocket;
a plurality of female buckles for buckling ECG electrodes are disposed on an inner surface of the inner lining; and
both sides of the combination where the front piece and the back piece are sewn are provided with a first zipper for adjusting tightness.

2. The combination of claim 1, comprising a neck area and an abdomen area, wherein a second zipper extending from the neck area to the abdomen area is disposed in a middle line of the front piece of the combination.

3. The combination of claim 1, wherein both the inner lining of the front piece and the back piece are made of two different kinds of cloth.

4. The combination of claim 3, wherein the two different kinds of cloth comprise non-elastic cloth and elastic cloth.

5. The combination of claim 1, wherein an anti-skid plastic elastic band is disposed at a lower part of an inner side of both the front piece and the back piece of the combination.

6. The combination of claim 1, wherein each of the ten front-end modules of active electrodes comprises a first elastic pad, a signal processing PCB, and a metal mask; the bolts pass through the signal processing PCB, the elastic pad, and the inner lining of the front piece, and then are connected to the female buckles; the metal mask is filled with silica gel which covers the signal processing PCB; and
a second elastic pad is disposed at an outer side of the metal mask whereby increasing the tight pressure of electrodes on a wearer's body.

7. The combination of claim 1, wherein the ECG electrodes comprise an electrode cap, an anti-skid washer, a conductive pad, and an electrode buckle; the electrode cap is made from conductive gel or conductive cloth mixed with elastic materials; the anti-skid washer is made from soft gel; the conductive pad is made from conductive materials; the electrode buckle is formed by stamping conductive metals and fixed at the center of the conductive pad.
